(19) United States
(12) Patent Application Publication
(10) Pub. No.: US 2025/0331769 A1
(43) Pub. Date: Oct. 30, 2025

(54) Apparatus and Method of Mechanical Phenotyping of Knees

(71) Applicant: New York Society for the Relief of the Ruptured and Crippled, maintaining the Hospital for Special S, New York, NY (US)

(72) Inventors: Suzanne MAHER, Highland Lakes, NJ (US); Russell Frederick WARREN, Greenwich, CT (US); Scott Alan RODEO, New York, NY (US); Tony CHEN, Highland Park, NJ (US); Ashley Pekmezian, Melbourne Beach, FL (US)

(73) Assignee: New York Society for the Relief of the Ruptured and Crippled, maintaining the Hospital for Special S, New York, NY (US)

(21) Appl. No.: 18/870,098

(22) PCT Filed: Jun. 2, 2023
(86) PCT No.: PCT/US2023/067857

§ 371 Date: Nov. 27, 2024

Related U.S. Application Data
(60) Provisional application No. 63/348,229, filed on Jun. 2, 2022.
(60) Provisional application No. 63/484,518, filed on Feb. 12, 2023.

(51) Int. Cl. A61B 5/00; A61B 5/055; G06V 40/10

(52) U.S. Cl. CPC A61B 5/4585; A61B 5/055; G06V 40/10; A61B 2562/0247; G06V 2201/033

(57) ABSTRACT

A method of phenotyping a knee is disciosed. The method includes assigning the knee to a predetermined class based on a determined percentage of load through the meniscus of the knee through a defined range of motion. The method also includes determining a course of medical treatment for the knee based on the predetermined class the knee is assigned.

### CROSS-REFERENCE TO RELATED APPLICATIONS

This application claims the benefit of U.S. Provisional Patent Application No. 63/348,229, filed Jun. 2, 2022, the entire disclosure of which is hereby incorporated by reference in its entirety for all purposes.

This invention was made with government support under AR066635 and AR075523 awarded by the National Institute of Health. The government has certain rights in the invention.

### BACKGROUND

The present invention relates generally to a method of categorizing knees in orthopedics to determine a surgical strategy. In particular, the subject disclosure relates to a method for mechanical phenotyping of knees.

The meniscus is a commonly injured portion of a knee or knee joint and is the most operated upon orthopedic tissue, with over 700,000 meniscal surgeries done annually in the U.S. In many cases, repair of the meniscus is not possible, instead, removal of the damaged tissue occurs in a “partial meniscectomy” (PM) procedure. However, removal of damaged tissue causes redistribution of forces across the joint, to which cartilage cannot adapt. As such, many patients show signs of osteoarthritis (OA) within months after surgery.

Current methods to treat injured tissue in the knee joint, for example, include using orthopedic scaffolds to repair or replace an injured meniscus or cartilage in an effort to restore mechanical function however, these are rarely used clinically, in part because it is unclear which patients will benefit most from mechanical reinforcement. Therefore, there remains a need for a method to determine which patients would benefit from scaffolds or other mechanical reinforcements. Such a need is satisfied by the method of the subject disclosure.

### BRIEF SUMMARY OF THE DISCLOSURE

A method of phenotyping a knee is disclosed. The method includes assigning the knee to a predetermined class based on a determined percentage of load through the meniscus of the knee through a defined range of motion. The method also includes determining a course of medical treatment for the knee based on the predetermined class the knee is assigned.

In some examples, the predetermined class is chosen from cluster 1, cluster 2, cluster 3, or cluster 4. In some examples, the method further includes the step of determining the percentage of the load through the meniscus of the knee at a fixed position or through the defined range of motion. In some examples, the percentage meniscal load is equal to the sum of the forces through a meniscus region divided by the sum of the forces through the entire knee compartment. In some examples, the method further includes the step of moving the knee though the defined range of motion at a predefined frequency; in some examples the position of the knee does not change. In some examples, the predefined frequency is approximately 0.1-0.3 Hz at approximately 0 to 60 percent gait. In some examples, the predefined range of motion is 0 to 60 percent gait. In some examples, the step of determining a course of medical treatment for the knee based on the predetermined class the knee is assigned includes determining whether scaffolding should be coupled to the meniscus. In some examples, the method further includes the step of applying a load to the knee. In some examples, the load is approximately 175-2250N. In some examples, the time the load is applied is greater than 20 seconds. In some examples, the method further includes the step of securing a sensor to the knee. In some examples, the sensor has a diameter of approximately 0.008″-0.01″. In some examples, the sensor has a thickness of approximately 0.001-0.006″. In some examples, the sensor is secured to an ACL and posterior capsule of the knee.

In accordance with another exemplary embodiment, the subject disclosure provides a method of phenotyping a knee, the method comprising: applying a load to the knee; determining a percentage of the load through a meniscus of the knee through a defined range of motion; and assigning the knee to a predetermined classification based on the determined percentage of the load through the meniscus of the knee.

In accordance with another exemplary embodiment, the subject disclosure provides a method of phenotyping knees comprising: securing a sensor to a knee; applying a load of about 175-2250N to the knee; determining a percentage of the load through a meniscus of the knee as measured by the sensor while the knee moves through a defined range of motion of about 0 to 60 percent gait cycle at a predefined frequency of about 0.1-0.3 Hz, wherein the percentage of the load through the meniscus is equal to the sum of the forces through a meniscus region divided by the sum of the forces through the entire knee compartment; and assigning the knee to a predetermined classification consisting essentially of cluster 1, cluster 2, cluster 3 and cluster 4, based on the determined percentage of the load through the meniscus of the knee.

In accordance with another exemplary embodiment, the subject disclosure provides a method of treating a partial meniscectomy of the knee joint comprising: applying a load to the meniscus of the knee joint through one of the femur and tibia; determining a percentage of the load passing through the meniscus of the knee joint; assigning the knee joint to a predetermined classification based on the determined percentage of the load passing through the meniscus; and determining a course of medical treatment of the partial meniscectomy based on the assigned classification of the knee joint.

The method further includes determining a percentage of the load passing through the meniscus of the knee joint is determined while the knee joint moves through a range of motion, wherein the determining a percentage of the load passing through the meniscus of the knee joint is determined while the knee joint moves at a predefined frequency. The percentage of the load passing through the meniscus of the knee joint is determined while the knee joint moves at a predefined frequency.

The method further comprises applying a sensor to the knee joint to measure a load on a meniscus of the knee joint.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the subject disclosure is not limited to the precise arrangements and instrumentalities shown.

In the drawings:

FIG. 1 is a sagittal view and a coronal view of a knee;

FIG. 2 is a schematic view of a sensor attached to the knee;

FIG. 3 is a schematic view of a load being applied to the knee;

FIG. 4 is another schematic view of the sensor attached to the knee;

FIG. 5 is a graphic representation of Percent Meniscal Loading Calculation;

FIG. 6 is a graphical representation of Meniscal Loading Variability;

FIG. 7 is another graphical representation of Meniscal Loading Variability;

FIG. 8 is a graphical representation of a Cluster Representation of Meniscal Loading Variability;

FIG. 9 is a graphic representation of a Weighted Center of Contact Position of a knee;

FIG. 10 is graphic representation of Posterior Shift of Contact Force After ACLx;

FIG. 11 is a graphic representation of Increased Velocity of Contact at Heel Strike After ACLx;

FIG. 12 is a flow diagram of the method described herein; and

FIG. 13 is a flow diagram of another method described herein.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, 11%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

Orthopedic repair of a patient's knee, generally illustrated as reference number 10 is the drawings, is a common surgical procedure in the United States. As a key load-bearing structure in the knee 10, a patient's meniscus is commonly injured and is the most operated upon orthopedic tissue. Such operations, such as partial meniscectomy, result in biochemical compositional changes in joint tissue are evident as early as 6 months after PM. These changes are thought to be driven by a change in contact forces across the joint, as a section of a key load distributing tissue is removed.

A method of phenotyping a knee 10 is disclosed herein. The method includes assigning the knee to a predetermined class based on a determined percentage of load through the meniscus of the knee 10 through a defined range of motion and determining a course of medical treatment for the knee 10 based on the predetermined class the knee 10 is assigned.

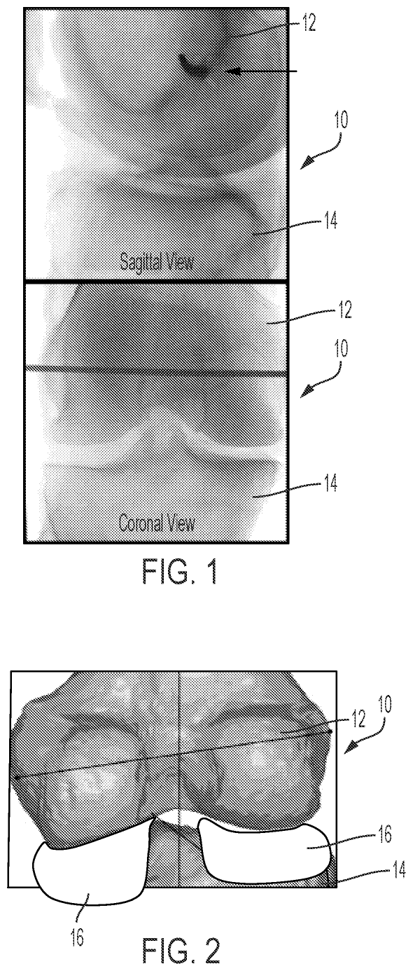

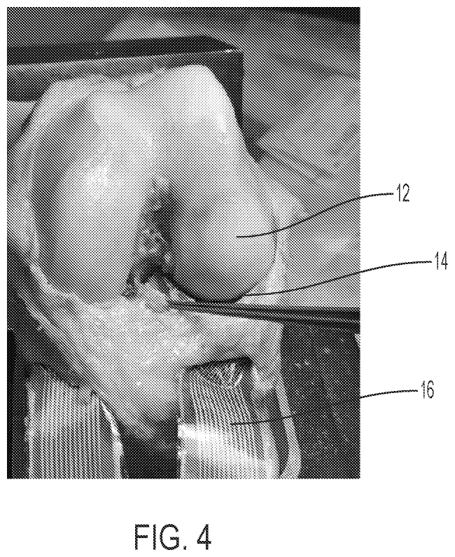

Referring now to the examples shown in FIGS. 1-13, in some examples, to assign the knee 10 to a predetermined class, the knee 10 may be prepared. In some examples, preparing the knee 10 may include striping the skin and other knee 10 tissue from a femur 12 and a tibia 14. Additionally, preparing the knee 10 may include pining the knee 10 though the epicondylar axis, as illustrated in FIG. 1. It is also contemplated that the knee 10 may be pinned through another location, if desired, that allows for natural range of motion of the knee joint. Once the knee 10 is prepared, the femur 12 and tibia 14 are secured. Securing may include any known femur 12 and tibia 14 securing methods including but not limited to potting, clamping and the like. In other examples, the knee may be prepared in other methods as desired.

Once the knee 10 is prepared, a sensor 16 may be inserted and secured to the knee 10, e.g., between the femur and tibia so as to measure contact forces applied by the distal condyles. In some examples, the sensor 16 is inserted using a telescopic insertion device, however, various other insertion devices have been contemplated. In some examples, the sensor 16 is configured to sense at least the following parameters of the knee joint contact forces: (i) peak contact force, (ii) contact area, (iii) percent force through the meniscus, (iv) weighted center of contact.

In some examples, the sensor 16 can be a thin electronic sensor containing an array of piezoelectric pressure sensing elements sealed within a thin plastic sheet. The sensor 16 may have any arrangement of electronic sensing elements including high density, medium density, or low density distribution. In some examples, the sensor 16 may have a diameter of approximately, 0.008″-0.01″, or the like. In some examples, the sensor 16 may have a thickness of approximately 0.001″-0.006″. In some examples, the sensor 16 can be a Tekscan, Inc Model 4010N, however, various other sensors have been contemplated.

In some examples, the sensor 16 is secured to the knee 10 to measure joint contact forces. More specifically, in some examples the sensor 16 can be configured to be secured to a PCL and posterior capsule of the knee 10. However, various other configurations have been contemplated.

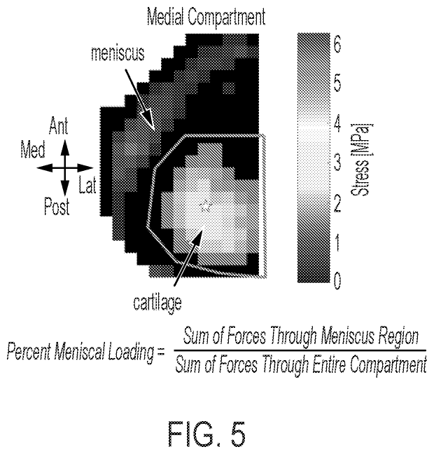

Further, in some examples, to assign the knee 10 to the predetermined class, a percentage of the load through the meniscus of the knee 10 through a defined range of motion is determined. In some examples, the percentage of the meniscal load through the defined range of motion is equal to a sum of the forces through a meniscus region divided by the sum of the forces through an entire knee 10 compartment, e.g., as illustrated in FIG. 5.

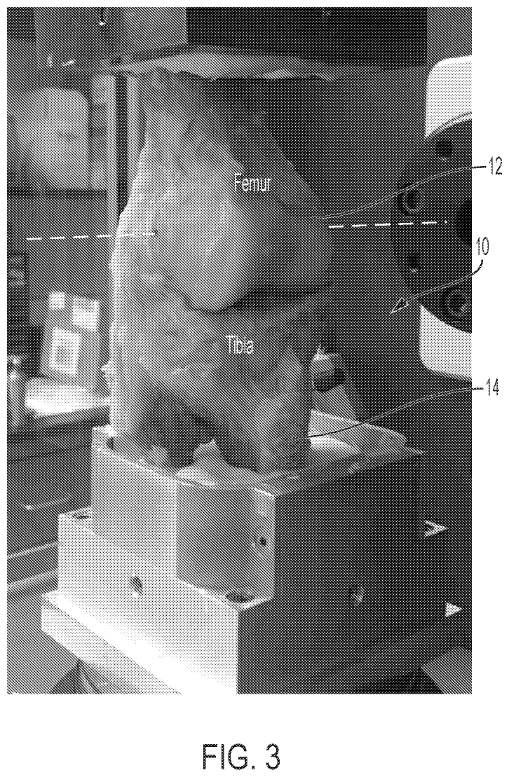

In some examples, to determine the percentage of the load through the meniscus of the knee 10 through a predefined range of motion, a load may be applied to the knee 10. In some examples, the load is approximately 200 N for a time of 30 seconds. However, other loads have been contemplated including but not limited to loads of about 100N-300N for a time of about 5 seconds to approximately 30 minutes. Additionally, in the example shown in FIG. 3, the load is a linear load applied along a longitudinal axis of both the femur 12 and the tibia 14 simultaneously. However, it is contemplated that the load may be perpendicular to the longitudinal axis of the femur 12 and tibia 14, a rotational load, and/or may be applied to only one of the femur 12 or tibia 14 at a time. In some examples, the load is applied by a testing apparatus such as a VIVO multi-axis testing apparatus. More specifically, in some examples the testing apparatus may be programmed to apply 50% of body weight in the axial direction.

In some examples, the load may be applied for a plurality of cycles of gait including but not limited to 10-15 cycles, 5-20 cycles, or 1-25 cycles.

In some examples, the defined range of motion at the predefined frequency is approximately 0 to 60 percent gait cycle at a frequency of approximately 0.2 Hz. It is also contemplated that the defined range of motion may include but is not limited to approximately 0 to 80 percent gait cycle, 0 to 70 percent gait, 0 to 50 percent gait cycle, or 0 to 40 percent gait cycle. Moreover, in some examples, the predefined frequency may include but is not limited to approximately 0.01-0.5 Hz.

In some examples, determining a percentage of the load through a meniscus of the knee step may be determined using a simulated stance method e.g., a patient in a stationary stance position, to gather the desired data. The simulated stance method can be used with or without the use of implanted sensors, e.g., a loaded Magnetic Resonance Imaging (MRI) technique wherein an MRI is taken with the knee joint having an applied predetermined axial load, such that data gathering is less invasive for the patient.

Classifications or clusters of knees can be based on e.g., percent load distribution through the meniscus while undergoing dynamic simulated gate, based on their demographic and geometric variable, based on percent force through the meniscal footprint while undergoing simulated gate, and/or based on lateral tibial spine height.

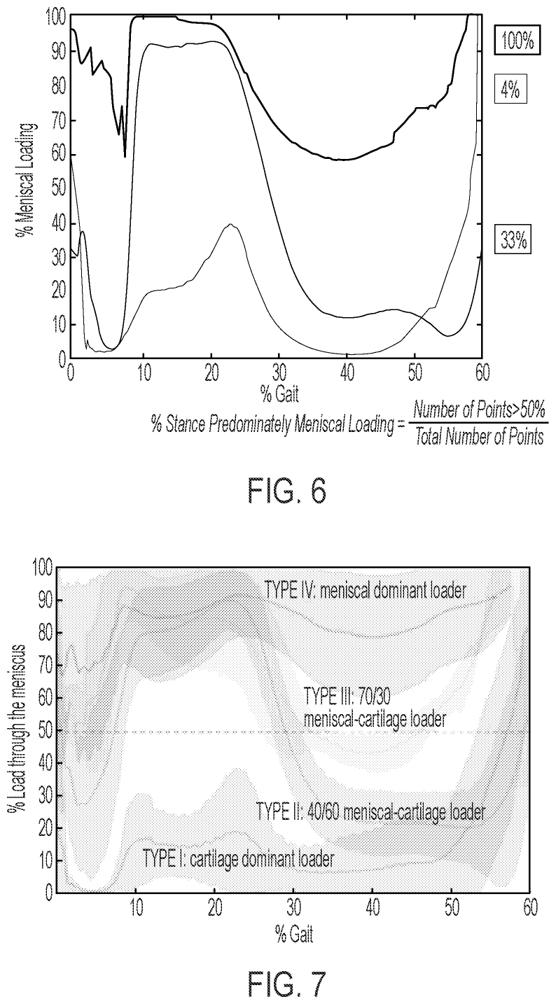

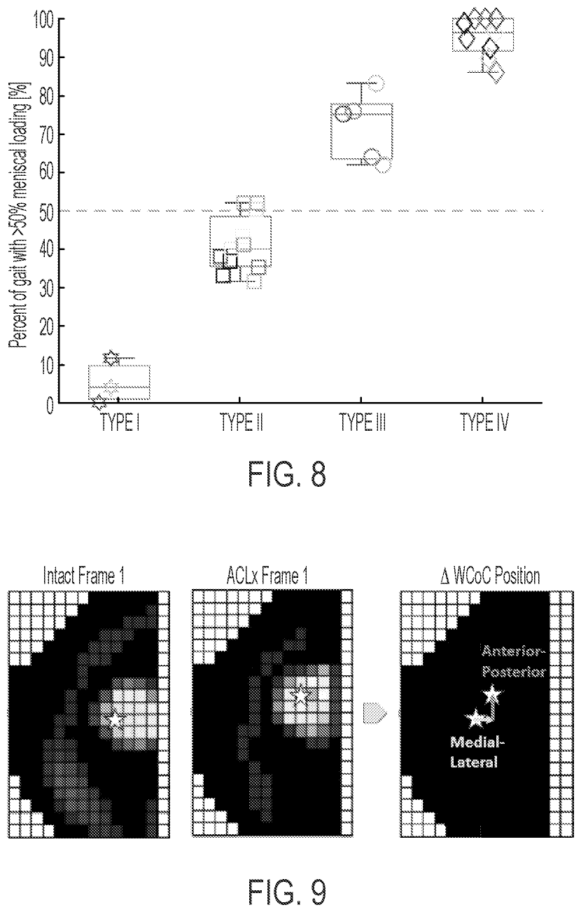

FIGS. 6-8 illustrate an exemplary example of mechanical data obtained from the method of phenotyping knees of the subject application based on cadaver studies. K-mean cluster optimization has been performed on data produced by obtaining percentage of load through the meniscus of the knee 10. The K-mean cluster optimization has been used to determine the existence of 4 types of knees 10 which are organized into predetermined classes or classifications, as illustrated in FIG. 8. These predetermined classes can be identified as Cluster 1, Cluster 2, Cluster 3, and Cluster 4. Cluster 1 is identified as a cartilage dominate loader. Cluster 2 is identified as a 40/60 meniscal-cartilage loader. Cluster 3 is identified as a 70/30 meniscal-cartilage loader. Cluster 4 is identified as a meniscal dominant loader. In Cluster 1, forces are distributed predominantly through regions of cartilage-to-cartilage contact, while in other knees 10, such as knees identified as Cluster 4, forces are distributed predominantly through menisci (also referred to as meniscal dominant loaders).

In alternative examples, the predetermined class or classifications can be adjusted such that the Clusters include two, three, five or more clusters. For example, the classes can be defined as clusters having various percentage meniscal-cartilage loader e.g., 30/70, 40/60, 50/50, 60/40, 70/30 meniscal-cartilage loader.

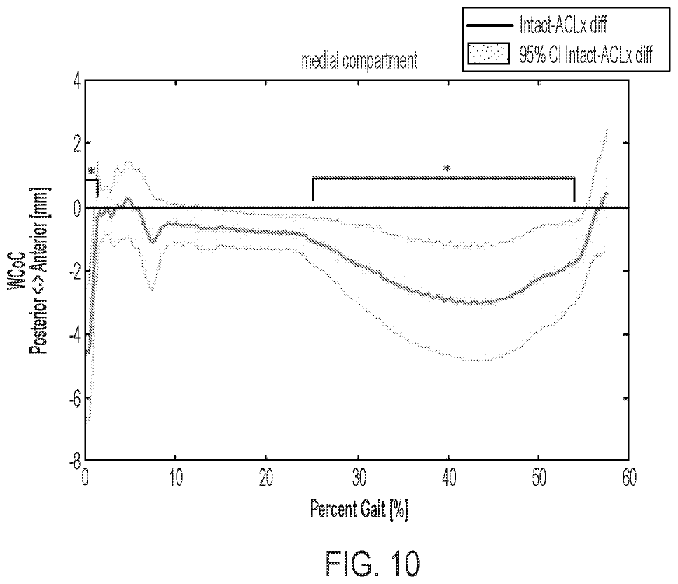

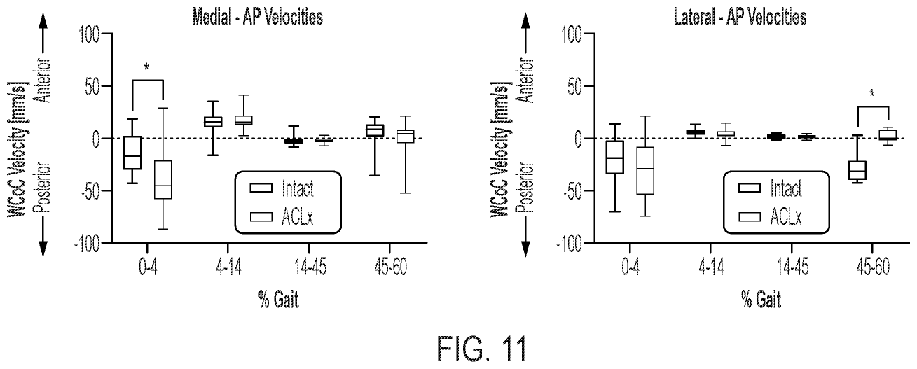

FIGS. 10 and 11 illustrate data on posterior shift of contact force after an Anterior Cruciate Ligament (ACL) transection and increased velocity of contact at heel strike after the ACL transection based on cadaver studies.

It has been found that when a partial meniscectomy is done, the magnitude of change in contact mechanics is not only associated with size and location of tissue removed, but dependent on the “type” of knee 10, i.e., the classification of the knee. Additional finding of the cadaver study include, for the medial compartment: posterior shift of contact at heel strike and mid-stance, increased velocity at heel strike. For the lateral compartment: decreased velocity at late-stance. The data further indicated that 39% of knees loaded the medial meniscus more after the ACL transection.

In some examples, an ACL transection is performed once the knee 10 has been moved through the defined range of motion at the predefined frequency. If an ACL transection is performed, the weighted geographic center of the knee may be moved to account for this transection, as illustrated in FIG. 9. In some examples, once the ACL transection is preformed, the applying load to the knee 10 and the moving the knee 10 through the defined range of motion steps are repeated and the predetermined class may be assigned or re-assigned based on the new data.

Once, the predetermined class of knee 10 has been assigned, a course of medical treatment for the knee 10 based on the predetermined class the knee 10 is determined. In some examples, determining the course of medical treatment may include determining whether scaffolding should be coupled to the meniscus. In some examples, the amount of scaffolding and location of scaffolding may also be determined based on the predetermined class of knee 10. In general, any applicable medical/surgical treatment can be applied which would benefit from an understanding of the mechanical phenotype of a patient's knee including but not limited to medical/surgical treatment of the ACL, cartilage, or other soft tissue.

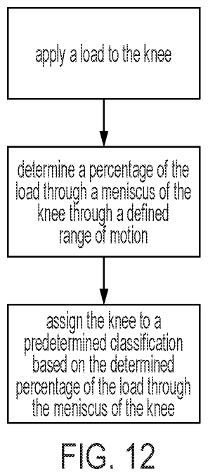

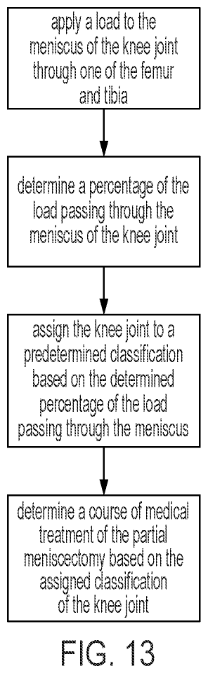

In operation, and as illustrated in FIGS. 12 and 13, the knee 10 is prepared and the sensor 16 is placed thereon. In some examples, the sensor 16 is placed below the meniscus, however, it is also contemplated that sensor 16 may be placed above or on the meniscus, as desired. Once the sensor 16 is secured, a load is applied to the knee 10 while the knee 10 is moving through the defined range of motion. In some examples, the load is applied to the meniscus of the knee joint through one of the femur and tibia. Next, the percentage of the load through the meniscus of the knee 10 through the defined range of motion is determined. The percentage of the load through the meniscus of the knee 10 is used to assign the knee 10 to the predetermined class. The predetermined class then is used to determine the course of medical treatment for the knee 10, e.g., such as whether scaffolding should be placed on the meniscus or not, and how much should be placed. In other examples, the predetermined class may be used to determine the course of medical treatment for other portions of the knee, including but not limited to medical treatment of the ACL, cartilage, or other soft tissue.

Scaffolds can be used to replace the removed tissue and restore mechanical function. By assigning each knee 10 to a predetermined class as described herein, the course of medical treatment can be specialized for each individual knee 10 type thereby providing specialized and improved overall care to the patient. Our findings show that some knees are “meniscal loaders” while other knee types substantially bypass the meniscus to load regions of cartilage-to-cartilage contact.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims.

### CLAUSES ACCORDING TO EXAMPLES OF THE DISCLOSURE

1. A method of phenotyping a knee, the method comprising:
applying a load to the knee;
determining a percentage of the load through a meniscus of the knee through a defined range of motion; and
assigning the knee to a predetermined classification based on the determined percentage of the load through the meniscus of the knee.

2. The method of clause 1, wherein the predetermined classifications consist essentially of cluster 1 or cluster 2.

3. The method of any of the above clauses, wherein the predetermined classifications consist essentially of cluster 1, cluster 2, cluster 3, and cluster 4.

4. The method of any of the above clauses, wherein the percentage of the load through the meniscus is equal to the sum of the forces through a meniscus region divided by the sum of the forces through the entire knee compartment.

5. The method of any of the above clauses, further comprising moving the knee though the defined range of motion at a predefined frequency.

6. The method of any of the above clauses, wherein the predefined frequency is about 0.1-0.3 Hz at about 0 to 60 percent gait cycle.

7. The method of any of the above clauses, wherein the defined range of motion is 0 to 60 percent gait cycle.

8. The method of any of the above clauses, wherein the load is about 175-2250N.

9. The method of any of the above clauses, wherein the time the load is applied is greater than 20 seconds.

10. The method of any of the above clauses, further comprising the step of securing a sensor to the knee.

11. The method of any of the above clauses, wherein the sensor has a diameter of approximately 0.008″-0.01″ or a thickness of approximately 0.001-0.006″.

12. The method of any of the above clauses, wherein the sensor is secured to an anterior cruciate ligament or a posterior capsule of the knee.

13. A method of treating a partial meniscectomy of the knee joint comprising:
applying a load to the meniscus of the knee joint through one of the femur and tibia;
determining a percentage of the load passing through the meniscus of the knee joint;
assigning the knee joint to a predetermined classification based on the determined percentage of the load passing through the meniscus; and
determining a course of medical treatment of the partial meniscectomy based on the assigned classification of the knee joint.

14. The method of any of the above clauses, wherein the determining a percentage of the load passing through the meniscus of the knee joint is determined while the knee joint moves through a range of motion.

15. The method of any of the above clauses, wherein the determining a percentage of the load passing through the meniscus of the knee joint is determined while the knee joint moves at a predefined frequency.

16. The method of any of the above clauses, further comprising applying a sensor to the knee joint to measure a load on a meniscus of the knee joint.

17. A method of phenotyping knees comprising:
securing a sensor to a knee;
applying a load of about 175-2250N to the knee;
determining a percentage of the load through a meniscus of the knee as measured by the sensor while the knee moves through a defined range of motion of about 0 to 60 percent gait cycle at a predefined frequency of about 0.1-0.3 Hz, wherein the percentage of the load through the meniscus is equal to the sum of the forces through a meniscus region divided by the sum of the forces through the entire knee compartment; and
assigning the knee to a predetermined classification consisting essentially of cluster 1 and cluster 2, based on the determined percentage of the load through the meniscus of the knee.

1. A method of phenotyping a knee, the method comprising:
applying a load to the knee;
determining a percentage of the load through a meniscus of the knee through a defined range of motion; and
assigning the knee to a predetermined classification based on the determined percentage of the load through the meniscus of the knee.

2. The method of claim 1, wherein the predetermined classifications consist essentially of cluster 1 or cluster 2.

3. The method of claim 1, wherein the predetermined classifications consist essentially of cluster 1, cluster 2, cluster 3, and cluster 4.

4. The method of claims 1, wherein the percentage of the load through the meniscus is equal to the sum of the forces through a meniscus region divided by the sum of the forces through the entire knee compartment.

5. The method of claims 1, further comprising moving the knee though the defined range of motion at a predefined frequency.

6. The method of claim 4, wherein the predefined frequency is about 0.1-0.3 Hz at about 0 to 60 percent gait cycle.

7. The method of claim 5, wherein the defined range of motion is 0 to 60 percent gait cycle.

8. The method of claims 1, wherein the load is about 175-2250N.

9. The method of claim 1, wherein the time the load is applied is greater than 20 seconds.

10. The method of claim 1, further comprising the step of securing a sensor to the knee.

11. The method of claim 10, wherein the sensor has a diameter of approximately 0.008″-0.01″ or a thickness of approximately 0.001-0.006″.

12. The method of claim 10, wherein the sensor is secured to an anterior cruciate ligament or a posterior capsule of the knee.

13. A method of treating a partial meniscectomy of the knee joint according to the method of claim 1 and further comprising the step of determining a course of medical treatment of the knee based on the assigned classification of the knee.

14. The method of claim 13, wherein the determining a percentage of the load passing through the meniscus of the knee joint is determined while the knee joint moves through a range of motion.

15. The method of claim 13, wherein the determining a percentage of the load passing through the meniscus of the knee joint is determined while the knee joint moves at a predefined frequency.

16. The method of claims 13, further comprising applying a sensor to the knee joint to measure a load on a meniscus of the knee joint.

17. The method of claim 1 further comprising the steps of securing a sensor to a knee; wherein the step of determining a percentage of the load through the meniscus of the knee is measured by the sensor while the knee moves through a defined range of motion of about 0 to 60 percent gait cycle at a predefined frequency of about 0.1-0.3 Hz, wherein the percentage of the load through the meniscus is equal to the sum of the forces through a meniscus region divided by the sum of the forces through the entire knee compartment, and wherein the predetermined classification consists essentially of cluster 1 and cluster 2.
